(19) United States
(12) Patent Application Publication
(10) Pub. No.: US 2020/0022715 A1
(43) Pub. Date: Jan. 23, 2020

(54) METHODS FOR REMOVING CALCIUM FROM PERIPHERAL ARTERY VESSELS

(71) Applicants: Ghassan S. Kassab, La Jolla, CA (US); Ali Dabiri, San Diego, CA (US)

(72) Inventors: Ghassan S. Kassab, La Jolla, CA (US); Ali Dabiri, San Diego, CA (US)

(21) Appl. No.: 16/516,230

(22) Filed: Jul. 18, 2019

Related U.S. Application Data
(60) Provisional application No. 62/700,105, filed on Jul. 18, 2018.

(51) Int. Cl. A61B 17/22; A61N 7/02

(52) U.S. Cl. CPC A61B 17/22012; A61N 7/02; A61B 2017/22079; A61B 2017/22025; A61B 2017/22014

(57) ABSTRACT

Methods of removing calcium and other deposits from peripheral and other vessel walls using ultrasound waves. In an exemplary method of treating a deposit in a patient's peripheral artery, the method comprises the steps of generating an ultrasound wave outside of the patient and directing the ultrasound wave toward a peripheral artery of the patient.

### PRIORITY

The present patent application is related to, and claims the priority benefit of, U.S. Provisional Patent Application Ser. No. 62/700,105, filed Jul. 18, 2018, the contents of which are hereby incorporated by reference in their entirety into this disclosure.

### BACKGROUND

Shockwave Medical, Inc. received FDA approval in June 2017 to disrupt calcified plaque using peripheral interventional balloon with Intravascular Lithotripsy (IVL) technology. The treatment is delivered on a standard balloon catheter platform where IVL technology combines the calcium disrupting power of lithotripsy with the familiarity of a balloon in a single enabling device. The catheter is equipped with ultrasound electrodes that break away the calcium deposit. This technology presently seems to be the state of art, but it is minimally invasive.

Shock wave methods have been used to crush kidney stones non-invasively by extracorporeal shock wave lithotripsy (ESWL) ureteroscopy. This method can be used to kidney stones as large as 2 cm in diameter. The five most common types of stones are comprised of calcium oxalate, calcium phosphate, uric acid, struvite, and cystine. Calcium oxalates, which are hard objects, constitute about 80% of human kidney stones. The patient is sedated or anesthetized and needs to lie down in the apparatus' bed. The patient's back is supported by a water-filled coupling device placed at the level of kidneys. A fluoroscopic x-ray imaging system or an ultrasound imaging system is used to locate the stone and aims the treatment. The number of shocks depends on the size of the kidney stone and can go as high as 2,500 shocks. The frequency is equal to the heart rate and the procedure takes about 45-60 minutes. The distance between the skin and the kidney is about 6-10 cm depending on the body mass index (BMI).

### BRIEF SUMMARY

In one embodiment a method of treating a deposit in a patient's peripheral artery comprises the steps of: generating an ultrasound wave outside of the patient; and directing the ultrasound wave toward a peripheral artery of the patient.

In another embodiment a method of treating a deposit in a patient's peripheral artery comprises the steps of: generating an ultrasound wave outside of the patient; directing the ultrasound wave toward a peripheral artery of the patient; introducing a suction catheter into the vasculature of the patient; and using the suction catheter to aspirate a fragmented part of the arterial deposit.

In one embodiment a method of treating a deposit in a patient's peripheral artery comprises the steps of: positioning an ultrasound wave source exterior to a patient; generating a plurality of ultrasound waves from the ultrasound wave source; and directing the plurality of ultrasound waves into the patient so that the deposit in the peripheral artery is treated.

In another embodiment the suction catheter comprises a flared tip.

In another embodiment a method of treating a deposit in a patient's peripheral artery comprises the steps of: generating a plurality of ultrasound waves outside of the patient; and directing the plurality of ultrasound waves toward the peripheral artery of the patient wherein the generated plurality ultrasound waves vary in power or rate of wave delivery.

In an alternate embodiment, the ultrasound waves have a rate of wave delivery of 2-4 Hz. In an alternate embodiment the ultrasound waves comprises a rectangular shaped focal beam. In another embodiment, the rectangular shaped focal beam has a length of 1-2 cm.

In another embodiment the method comprises the step of directing the plurality of ultrasound waves so that a length of the peripheral artery is treated. In another embodiment the ultrasound wave source travels along the length of the artery every few minutes to treat a desired length of the artery.

In another embodiment of a method of treating a deposit in a patient's peripheral artery using an exemplary system of the present invention, the system comprising an ultrasound wave source and a suction catheter, the method comprises the steps of: emitting a plurality of ultrasound waves from the ultrasound wave source; placing the ultrasound wave source exterior to and facing the patient so the plurality of ultrasound waves travel into the patient artery; and introducing the suction catheter into the vasculature of the patient and aspirating fragmented deposit parts.

In another embodiment, the method of comprises the step of manipulating the ultrasound wave source so that a length of the artery is treated by the plurality of ultrasound waves.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 shows a schematic view according to an exemplary embodiment of the present disclosure; and

FIG. 2 shows a close up view of a peripheral artery from FIG. 1, according to an exemplary embodiment of the present disclosure.

An overview of the features, functions and/or configurations of the components depicted in the various figures will now be presented. It should be appreciated that not all of the features

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

### Principle of Operation of the ESWL Technology

By introducing high power ultrasound into a liquid medium, the sound waves are transmitted in the fluid and create alternating high-pressure (compression) and low-pressure (rarefaction) cycles, with rates depending on the frequency of the ultrasound wave. During the low-pressure cycle, high-intensity ultrasonic waves create small vacuum bubbles or voids in the liquid. When the bubbles attain a volume at which they can no longer absorb energy, they collapse violently during a high-pressure cycle. This phenomenon is termed cavitation. During the implosion, very high temperatures (˜5,000K) and pressures (˜2,000 atm) are reached locally. The transient collapse of the bubbles that gives rise to local temperature and pressure maxima is at the root of the observed effects of ultrasound on objects in front of it. This negative pressure drives cavitation bubble activity that is critical to stone comminution. It is also intense compressive wave that induces mechanical forces inside the stone that may lead to fragmentation, most likely by a spall mechanism. Efficient transfer of acoustic energy from one medium to another only occurs when the acoustic impedances are very close. A water/tissue interface results in very good coupling, and theoretically, it should be possible to transfer more than 99% of the energy of the shock wave into the body. But the presence of even a small pocket of air at the skin surface will result in a dramatic reduction in energy transfer to the patient. Thus, the manner in which the shock wave is coupled to the body is critical. The “first-generation” lithotriptors were electrohydraulic lithotriptors and used an open water bath in which the patient was immersed. Thus, there was nothing but water between the shock source and the patient. This is ideal except that the bubbles that drift up from the spark-gap, or the cavitation bubbles that form along the path of the shock wave, have the potential to collect against the skin of the patient and interfere with the propagation of subsequent shock waves. Most current lithotriptors have the shock wave source mounted in a “therapy head,” which is filled with water. The therapy head is capped by a thin rubber membrane pressed against the patient and through which the shock wave passes. A coupling agent such as gel or oil is smoothed on the rubber membrane and the patient's skin to ensure good coupling by reducing air pockets. There are three main parameters of shock wave delivery for a lithotriptor (power, number of shock waves and rate of shock wave delivery) can be adjusted to ensure equivalency between different machines. The rate of shock wave delivery ranges between 2-4 Hz. At 4 Hz operation, it takes about 10 minutes to break down kidney stones of 10-20 mm in diameter in a typical treatment.

### Proposed Non-Invasive Solution

In one embodiment of the invention, ESWL technology is used to non-invasively pulverize the calcium deposits in the arteries. The average thickness of calcium build-up in the various peripheral arteries is significantly less than the average kidney stone diameter. The skin depth of the peripheral arteries is not very different from the skin depth of the kidney. It may be that the calcium deposit can be broken down in much less time than the kidney stone if we obtain the same energy coupling between the artery and the shockwave. The ultrasound wave source can be built in such a way to create a rectangular shaped focal beam with 1-2 cm length. The ultrasound transducer can move along the artery every few minutes to cover the total desired length (may be up to 20 cm). The time required at each location will depend on the thickness of calcium to be disrupted. A suction catheter (with flared tip 26) can be used to aspirate the fragmented calcium particles during the ESWL procedure if calcium deposits are not sufficiently pulverized.

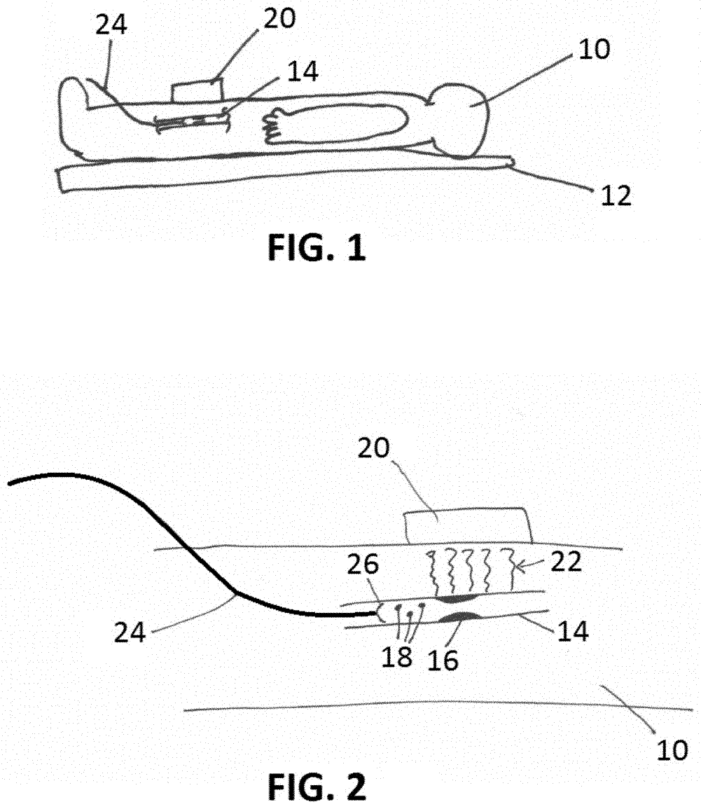

As shown in FIGS. 1-2, a system for treatment may comprise at least an ultrasound wave source 20 and a suction catheter 24.

An exemplary embodiment of a method for treatment may generally include the steps as follows. The patient 10 to be treated can lay on a supporting surface such as a bed or operating table 12. An ultrasound wave source 20 is placed exterior to the patient 10, such as touching the patient's skin and over the area of the body that contains the vessel 14 having the deposit 16 to be treated. Ultrasound waves 22 are then generated from the source 20 and travel into the patient 10 to interact with the deposit 16 in the vessel 14. The waves 22 ultimately break up the deposit 16 into smaller parts 18. If desired the waves can be directed along the length of the vessel 14, such as where there are more than one deposit 16 or lengthy deposits 16.

This procedure can be paired with a suction catheter 24. A suction catheter 24 is introduced into the vessel 14 to be treated, at or near the site of the deposit 16. As the ultrasound waves 22 interact with the deposit 16, parts of the deposit 18 will loosen and enter the bloodstream. The suction catheter 24 can be activated to aspirate, or collect the loose deposit particles 18.

While various embodiments of devices and methods for treating a deposit in a peripheral vessel of the present invention have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A method of treating a deposit in a patient's peripheral artery, comprising the steps of:
generating an ultrasound wave outside of the patient; and
directing the ultrasound wave toward a peripheral artery of the patient.

2. The method of claim 1, further comprising the steps of:
introducing a suction catheter into the vasculature of the patient; and
using the suction catheter to aspirate a fragmented part of the arterial deposit.

3. The method of claim 1, further comprising the steps of:
generating a plurality of ultrasound waves outside of the patient; and
directing the plurality of ultrasound waves toward the peripheral artery of the patient wherein the generated plurality ultrasound waves vary in power or rate of wave delivery.

4. The method of claim 3, wherein the plurality of ultrasound waves have a rate of wave delivery of 2-4 Hz.

5. The method of claim 1, wherein the ultrasound wave comprises a rectangular shaped focal beam.

6. The method of claim 1, wherein the rectangular shaped focal beam has a length of 1-2 cm.

7. The method of claim 3, further comprising the step of:
directing the plurality of ultrasound waves so that a length of the peripheral artery is treated.

8. The method of claim 2, wherein the suction catheter comprises a flared tip.

9. The method of claim 1, wherein the ultrasound wave is generated from an ultrasound wave source positioned outside the patient.

10. A method of treating a deposit in a patient's peripheral artery, comprising the steps of:
positioning an ultrasound wave source exterior to a patient;
generating a plurality of ultrasound waves from the ultrasound wave source; and
directing the plurality of ultrasound waves into the patient so that the deposit in the peripheral artery is treated.

11. The method of claim 10, wherein the ultrasound wave source generates a rectangular shaped focal beam.

12. The method of claim 11, wherein the rectangular shaped focal beam is 1-2 cm in length.

13. The method of claim 10, further comprising the step of:
directing the plurality of ultrasound waves so that a length of the peripheral artery is treated.

14. The method of claim 13, wherein the ultrasound wave source travels along the length of the artery every few minutes to treat a desired length of the artery.

15. The method of claim 10, wherein the plurality of ultrasound waves have a rate of wave delivery of 2-4 Hz.

16. A method of treating a deposit in a patient's peripheral artery using system comprising an ultrasound wave source and a suction catheter, the method comprising the steps of:
emitting a plurality of ultrasound waves from the ultrasound wave source;
placing the ultrasound wave source exterior to and facing the patient so the plurality of ultrasound waves travel into the patient artery; and
introducing the suction catheter into the vasculature of the patient and aspirating fragmented deposit parts.

17. The method of claim 16, further comprising the step of:
manipulating the ultrasound wave source so that a length of the artery is treated by the plurality of ultrasound waves.

18. The method of claim 16, wherein the plurality of ultrasound waves have a rate of wave delivery of 2-4 Hz.

19. The method of claim 16, wherein the ultrasound wave source is configured to emit a rectangular shaped focal beam.

20. The method of claim 19, wherein the rectangular shaped focal beam has a length of 1-2 cm.
